(19) United States
(12) Patent Application Publication
(10) Pub. No.: US 2004/0081691 A1
(43) Pub. Date: Apr. 29, 2004

(54) Granules containing a plant substance and process for preparing them

(75) Inventors: Patrice Debregeas, Paris (FR); Gerard Leduc, Malesherbes (FR); Domingo Bernabe, Daumont (FR)

Correspondence Address: FOLEY AND LARDNER, SUITE 500, 3000 K STREET NW, WASHINGTON, DC, 20007, US

(73) Assignee: D B F

(21) Appl. No.: 10/689,469

(22) Filed: Oct. 20, 2003

Related U.S. Application Data
(62) Division of application No. 09/312,485, filed on May 17, 1999.

(30) Foreign Application Priority Data
Mar. 12, 1999 (FR) FR99 03075

(51) Int. Cl. A61K 9/20; A61K 35/78

(52) U.S. Cl. 424/465; 424/725

(57) ABSTRACT

The present invention relates to granules containing at least one plant substance, characterized in that they each comprise a neutral core having a particle size of between 200 and 1600 μm coated with a layer containing the plant substance combined with a pharmaceutically acceptable excipient. These granules may be obtained from a plant extract in the form of a dry, fluid or soft extract, by powder-coating or by coating in solution.

### CROSS-REFERENCE TO RELATED PATENT APPLICATIONS

This application is a divisional of application Ser. No. 09/312,485, filed May 17, 1999, which claims the benefit of French patent application FR 99 03075, filed Mar. 12, 1999. The aforementioned applications are incorporated by reference in their entireties.

### BACKGROUND

The subject of the present invention is a new formulation in the form of granules containing a plant substance as well as the process for preparing it.

More precisely, the present invention relates to granules containing at least one plant substance and each comprising a neutral core coated with a layer containing the said plant substance combined with a pharmaceutically acceptable excipient.

The formulations containing plant substances which are already described in the prior art are in the form of powders, granules, tablets or oral solutions.

The major problem with formulations in powdered form is that the plant powder has to be mixed with excipients which are also in powdered form. A mixture of powders is then obtained which is hardly homogeneous and reproducible.

Furthermore, powders are very hygroscopic and they therefore pump moisture from the granules and from the gelatin capsule, which become brittle. This poses problems of stability, and the proportion in the gelatin capsule is not homogeneous.

This problem is solved within the framework of the present invention because, in the case of the application of a plant substance in the form of a dry extract onto neutral micrograms, there is no mixing of powder but the application of the dry extract onto neutral granules with excipients which are not powders.

The granules according to the invention have the advantage of being easier to package into gelatin capsules than powders of being more stable to storage than the formulations of the prior art and of having a reproducible proportion.

As for tablets, they have the same problems as powders. Moreover, plant extracts are not always compressible and compressing agents are not always authorized in the food industry.

Finally, the oral fluid forms are often bitter and foul-smelling to the extent that sweeteners and stabilizers need to be added. In addition, the oral fluid forms may exhibit physical or chemical instability during storage, a low content of characteristic plant constituents, and frequently contain ethyl alcohol in a moderately large quantity, which is not generally desirable for the oral administration of medicinal products.

The multiparticulate form of the formulation of the invention makes it possible to obtain a uniform and reproducible release profile.

In addition, the granules of the invention which each contain a layer of plant substance mounted on a neutral core may be coated with an outer layer so as to modify their properties. The outer layer comprises, for example, an enteric polymer, a polymer intended to prolong the release of the plant substance or a polymer intended to mask the taste or the odour of the plant substance.

The formulation according to the invention has the advantage of being stable during storage, of having an enhanced bioavailability, and of being able to integrate high doses of plant substance.

FR 2,721,512 describes a process for the preparation of granules by extrusion-spheronization from a polymer with absorbent or adsorbent properties. The polymer is sprayed with an aqueous-alcoholic fluid plant extract.

The synthetic or natural polymer is optionally combined with auxiliary substances, such as lactose or PVP, which make it possible to modulate the porosity of the spheroids and their rate of dissolution.

The extrusion-spheronization technique has many disadvantages: it requires the addition of a quantity of water at least equal to the quantity of excipients, the granules obtained by this technique have high moisture levels and their drying takes too long. In addition, the process described in FR 2,721,512 uses powders.

FR 2,616,068 describes a process which consists in granulating a dry or soft plant extract with methyl cellulose or silica.

FR 2,682,874 describes a process for the preparation of an extract of active ingredient in dry form from a fluid extract, which consists in adsorbing an aqueous-alcoholic solution of the active ingredient onto porous grains of cellulose or silica. The grains have a particle size which is in the micron range. These grains are then adsorbed onto porous granules 0.1 to 0.5 mm in diameter, which for example consist of sugar.

FR 2,737,134 describes a process which consists in coating cores, having a diameter of less than 0.01 mm, consisting of maltisorb or of a sodium bicarbonate/citrate mixture, with a compound in powdered form and a compound in solution. The compound in solution is an essential oil and/or a concentrated fluid plant extract.

### SUMMARY

The subject of the present invention is granules which overcome the disadvantages of the prior art formulations. These granules containing at least one plant substance are characterized in that they each comprise a neutral core having a particle size of between 200 and 1600 μm coated with a layer containing the plant substance combined with a pharmaceutically acceptable excipient.

The plant substance may be derived from plants chosen from garlic, Echinacea, Ginkgo biloba, ginseng, Harpagophytum, kava, St.-John's-wort, green tea, valerian, Missouri grape, artichoke, hawthorn, burdock, birch, alder buckthorn, blackcurrant, blessed thistle, Fucus, Hamamelis, horse chestnut, balm, Orthosiphon, passion flower, dandelion, horsetail, meadowsweet, sage, spirulina and mixtures thereof.

The neutral core consists of a substance chosen from sugar, starch, mannitol, sorbitol, xylitol, cellulose, talc and mixtures thereof.

The neutral cores may also consist of a starch/sucrose core in 20/80 mass ratios which is coated with 80% by weight of starch. In such neutral cores, the proportion by mass of sugar is advantageously less than 20%.

The layer containing the plant substance may contain a binder. A sugar such as sucrose, polyvinylpyrrolidone, lac gum or hydroxypropylmethyl-cellulose is advantageously used as binder.

### DETAILED DESCRIPTION OF THE PREFERRED EMBODIMENTS

The granules according to the invention may consist of a neutral core coated with a layer containing the plant substance, itself coated with an outer layer intended to mask the taste and/or the odour of the plant substance, to delay its release or to control its release.

When the outer layer is intended to control the release of the plant substance, it advantageously contains lac gum, PVP, a copolymer of methacrylic acid (Eudragit®) or of Aquacoat® with a plasticizer.

As polymer intended to mask the taste and/or the odour of the plant substance, a copolymer of methacrylic acid (Eudragit NE 30D® or Eudragit E 100®) or hydroxypropylmethylcellulose (Pharmacoat®) may be used.

It is also possible to use, as enteric polymer, lac gum by spraying an alcoholic solution containing 10% by weight of lac gum. At higher concentrations, between 20 and 40%, lac gum fulfils the function of a delayed-release polymer.

In the granules, the content of plant substance is between 0.1 mg/g and 750 mg/g.

The present invention relates in particular to garlic granules with masked odour and taste, Ginkgo biloba granules, one daily dose, prolonged-release ginseng granules, enteric Harpagophytum granules, prolonged-release green tea granules, prolonged-release Orthosiphon granules, valerian granules with masked taste and odour and prolonged-release St.-John's-wort granules.

The present invention also relates to a process for the preparation of the granules described above.

The process according to the invention allows better reproducibility of the proportion; it also makes it possible to formulate the plant substance from a dry, soft or fluid extract.

The granules according to the invention may contain several plant substances used in the form, independently of each other, of a fluid, dry or soft extract.

According to the definition given in the pharmacopoeia, plant extracts are concentrated preparations which are liquid, solid or of intermediate consistency, generally obtained from dried plant raw materials. For some preparations, the materials to be extracted may undergo a preliminary treatment (such as inactivation of enzymes, grinding or defatting).

Fluid extracts are liquid preparations of which, in general, a portion by mass or by volume corresponds to a portion by mass of dried raw material. These preparations are adjusted, if necessary, so as to meet the requirements of content of solvents, of constituents or of dry residue.

Soft extracts are preparations having an intermediate consistency between fluid extracts and dry extracts. Soft extracts are prepared by partial evaporation of the solvent which served for their preparation. Only ethanol at an appropriate titre or water are used. Soft extracts have in general a dry residue which is not less than 70 percent m/m. They may contain appropriate antimicrobial preservatives.

Dry extracts are solid preparations obtained by evaporation of the solvent which served for their production. Dry extracts have in general a dry residue which is not less than 95 percent m/m. Appropriate inert substances may be added.

According to the process of the invention, the granules are obtained by powder-coating when the plant substance is in the form of a dry extract.

Powder-coating is advantageously carried out by alternately spraying an alcoholic or aqueous-alcoholic solution of a binder, and the dry extract.

The granules are obtained by coating in solution when the plant substance is in the form of a soft or fluid extract.

In the case of a fluid extract, the active layer may be coated with a layer obtained by spraying a solution of a binder. The fluid extract preferably contains about 30 to 40% alcohol.

The process according to the invention advantageously makes it possible to limit the quantity of organic solvent used. During the process of the invention, 5 to 25% by weight of organic solvents are used.

The size of the granules used will be chosen as a function of the type of extract used and as a function of the desired proportion.

The size of the Neutres is between 950 and 1400 μm, when the plant extract is dry.

The size of the Neutres is between 900 and 1250 μm, when the plant extract is soft or fluid.

The percentage by mass of extract for the fluid extract used in the process of the invention is advantageously between 15 and 25% relative to the weight of the granules.

The percentage by mass of extract for a dry extract may be as high as 75% relative to the weight of the granules; it is preferably between 35 and 55%.

The granules according to the invention are prepared according to coating techniques known in the art, preferably in a pan or in a fluidized air bed.

The invention is illustrated without any limitation by the following examples.

### EXAMPLE 1

Green tea granules are prepared according to the following sequence of steps in a conventional pan. The green tea is in the form of a dry extract.

| | QUANTITY (KG) |
|---|---|
| Neutres | 32.5-33.5 |
| Coating | |
| Dry extract of green tea | 40.5-41.5 |
| PVP at 20% in alcohol | 14-20 |
| Precoating | |
| PVP at 20% in alcohol | 4 |
| Talc | 1.6 |
| Lubrication | |
| Talc | 0.1 |

The Neutres used have a particle size of between 0.800 and 1.000 mm.

The green tea coating step may be carried out in a single stage or in several stages by alternately spraying the plant extract and a solution of polyvinylpyrrolidone (PVP K30®) at 20% in ethanol.

During the coating, precoating and lubricating steps, the granules are sieved at 1.0-1.18 mm, 1.18-1.25 mm and 1.18-1.25 mm, respectively, and then dried for 8 hours, respectively at room temperature and 30° C.

Granules of the following formula are obtained:

| | P rcentage by mass |
|---|---|
| Dry extract of green tea | 49.9-52.3 |
| Neutres | 40.0-42.2 |
| PVP K30 ® | 4.5-6.7 |
| Talc | 2-2.2 |

Their water content is of the order of 0.7-1.5% by mass.

### EXAMPLE 2

| RAW MATERIALS | PERCENTAGE BY MASS |
|---|---|
| Neutres | 39.9 |
| Dry extract of Harpagophytum | 35.4 |
| PVP K30 | 2.6 |
| BDLG* | 2.2 |
| Alcohol 95% | 19.4 |
| Talc | 0.5 |

*BDLG: Bleached dewaxed lac gum.

The Neutres have a particle size of between 800 and 1000 microns.

The Neutres and the plant extract are sprayed with an alcoholic solution of polyvinylpyrrolidone. The granules are sieved and dried. During a second step, a layer of lac gum is applied still using an alcohol solution of polyvinylpyrrolidone.

The granules are again sieved and dried.

Finally, the granules are lubricated with talc.

### EXAMPLE 3

The granules having the following composition are prepared:

| RAW MATERIALS | PERCENTAGE BY MASS |
|---|---|
| Fluid extract of Harpagophyturn | 18.5 |
| Neutres | 67.4 |
| Fine crystalline sucrose | 4.1 |
| Purified water | 4.1 |
| Alcohol | 5.2 |
| Talc | 0.7 |

according to the process described below.

The Neutres are introduced into the tank and the fluid extract is sprayed in fractions. The granules are sized by sieving and then dried under an air bed. A 33% sucrose solution in an ethanol/water mixture is then applied. The granules are again sieved and dried, and then lubricated with talc.

### EXAMPLE 4

| RAW MATERIALS | PERCENTAGE BY MASS |
|---|---|
| Neutres | 41.9 |
| Dry extract of Ginkgo biloba | 30.4 |
| PVP K30 ® | 5.5 |
| Alcohol 95% | 21.9 |
| Talc | 0.3 |

### EXAMPLE 5

| RAW MATERIALS | PERCENTAGE BY MASS |
|---|---|
| Fluid extract of Ginkgo biloba | 19.2 |
| Neutres | 61.5 |
| PVP K30 ® | 3.0 |
| Alcohol 95% | 12.3 |
| Talc | 4.0 |

The Neutres are introduced into the tank and the fluid extract is sprayed in fractions. The granules are sized by sieving and then dried under an air bed. A solution of polyvinylpyrrolidone in alcohol is then applied. The granules are again sieved and dried, and then lubricated with talc.

1. Granules containing at least one plant substance, comprising a neutral core having a particle size of between 200 and 1600 μm coated with a layer containing the plant substance combined with a pharmaceutically acceptable excipient, wherein the layer containing the plant substance further comprises polyvinylpyrrolidone as a binder.

2. Granules according to claim 1, wherein the neutral core consists of a substance selected from the group consisting of sugar, starch, mannitol, sorbitol, xylitol, cellulose, talc, and mixtures thereof.

3. Granules according to claim 1, wherein the neutral core consists of a starch/sucrose core in a 20/80 mass ratio, which is coated with 80% by weight of starch.

4. Granules according to claim 1, wherein the pharmaceutically acceptable excipient used in the layer containing the plant substance is prepared from a solution of polyvinylpyrrolidine.

5. Granules according to claim 1, wherein the layer containing the plant substance is coated with an outer layer capable of masking the taste or odor of the plant substance.

6. Granules according to claim 5, wherein the outer layer is capable of controlling the release of the plant substance and contains lac gum, PVP, a copolymer of methacrylic acid or a colloidal dispersion of ethylcellulose with a plasticizer.

7. Granules according to claim 5, wherein the outer layer is capable of delaying the release of the plant substance and contains a copolymer of methacrylic acid, lac gum or a colloidal dispersion of ethylcellulose with a plasticizer.

8. Granules according to claim 5, wherein the outer layer is capable of masking the taste or odor of the plant substance and contains a copolymer of methacrylic acid or hydroxypropylmethylcellulose.

9. Granules according to claim 1, wherein the plant substance is selected from the group consisting of garlic, Echinacea, Ginko biloba, ginseng, Harpagpphytum, kava, St.-John's-wort, green tea, valerian, Missouri grape, artichoke, hawthorn, burdock, birch, alder buckthorn, blackcurrant, blessed thistle, Fucus, Hamamelis, horse chestnut, balm, Orthosiphon, passion flower, dandelion, horsetail, meadowsweet, sage, spirulina and mixtures thereof.

10. Granules according to claim 1, wherein the content of plant substance is between 0.1 mg/g and 750 mg/g weight of plant substance to the total weight of the granule.

11. Granules according to claim 1, wherein the layer containing the plant substance further comprises sucrose, lac gum, hydroxypropylmethylcellulose, or combinations thereof as a binder.

12. Granules containing at least one plant substance, comprising:
a neutral core having a particle size of between 200 and 1600 μm; and
a coating formed by applying,
(A) one or more layers which comprise a homogenous mixture of a plant substance and a solution including a pharmaceutically acceptable excipient, wherein the layer further comprises polyvinylpyrrolidone as a binder; or
(B) alternating layers, wherein one or more layers comprise the plant substance, and one or more layers comprise the solution including the pharmaceutically acceptable excipient, wherein the layer that comprises the solution including the pharmaceutically acceptable excipient further comprises polyvinylpyrrolidone as a binder.
